(19) United States
(12) Patent Application Publication
(10) Pub. No.: US 2025/0215398 A1
(43) Pub. Date: Jul. 3, 2025

(54) CELL CULTURE APPLICATION METHODS OF USING A SEPARATION WELL MICROPLATE

(71) Applicants: MOLECULAR DEVICES (AUSTRIA) GMBH, Puch Bei Hallein (AT); MOLECULAR DEVICES LLC, San Jose, CA (US)

(72) Inventors: Josef Atzler, Hallein (AT); Sara Sofia Deville, Salzburg (AT); Oksana Sirenko, San Carlos, CA (US); Felix Spira, Neumarkt am Wallersee (AT)

(21) Appl. No.: 18/848,057

(22) PCT Filed: Mar. 17, 2023
(86) PCT No.: PCT/US2023/064645

§ 371 Date: Sep. 17, 2024

Related U.S. Application Data
(60) Provisional application No. 63/321,373, filed on Mar. 18, 2022.

(51) Int. Cl. C12N 5/071; C12M 1/00; C12M 1/12; C12M 3/00; C12N 5/0775

(52) U.S. Cl. CPC C12N 5/0697; C12M 21/08; C12M 25/04; C12M 27/20; C12N 5/0662; C12N 2502/1323; C12N 2513/00; C12N 2533/90

(57) ABSTRACT

Automatable methods of horizontal co-culturing of cells using a single separation well microplate are provided. The disclosure also provides improved methods of quantifying secreted factors from cultivated cells, and methods for isolating target cells from organoids in a single separation well microplate.

### CROSS-REFERENCE TO RELATED APPLICATIONS

This application is the national phase of PCT International Application No. PCT/US2023/064645 filed on Mar. 17, 2023, which in turn claims the benefit of and priority to U.S. Provisional Application No. 63/321,373, filed Mar. 18, 2022, the entire contents of which are incorporated herein by reference.

### BACKGROUND

Culturing cells in a three-dimensional (3D) environment yields cellular behavior and morphology that more closely matches what is observed in the human body. 3D hydrogels/hydroscaffolds used for this kind of culturing have a unique attribute: cells can be deposited in specific locations in 3D space and remain in position for extended time periods. This enables the creation of structures (e.g., spheroids, tumoroids, organoids, and/or other multi-cellular bodies) and co-culture environments where cellular interactions and developments over time are observed.

So far, trans wells are typically needed for vertical co-culture. However, the handling of trans wells may be relatively complicated and difficult to automate, and cells are difficult to image. The supernatant can be transferred to a different detection plate using ELISA or a different method to analyze. However, kinetic measurements and direct comparison of different hormones secreted by organoids is not practical so far. Capturing of adult stem cells is possible so far if an additional capture plate is used for washing.

For in vitro toxicity studies there is often a need to have several cell types present: for example, cardiac cells and liver cells, which would metabolize chemicals, neurons and astrocytes, cardiac cells and endothelial cells, intestinal cells and liver cells, and the like. While co-culture of different cells or transfer of supernatants are current solutions, obtaining clean data can be difficult because of the need to distinguish between different cells, or evaluation of effects from small amounts of secreted factors.

Methods of using a single separation well microplate to allow not only physical separation while allowing fluid communication between different cell types, but also easy detection and evaluation of effects over time are desirable. Methods of using a single separation well microplate to allow evaluation of an in vivo-type situation combining immune cell migration, infiltration, and target tumor cell killing in one assay are desirable.

### SUMMARY

Methods are provided for co-culture of different cell types in separate compartments that allow efficient cross-talk and interaction of cell types and tissues, including but not limited to cell migration, neurite growth, angiogenesis, immune cell migration and killing, metastasis, cell invasion, monocyte adhesion, apoptosis, cell differentiation, stem cell implantation, inflammation, and secretion of multiple growth factors and metabolites.

The disclosure provides methods for using separation well microplates supporting horizontal co-culturing in an automation-friendly way. Separation well microplates support culturing of different cell types in adjacent wells that can be hydraulically connected using, for example, a pipette as a pump, or connected by laminar flow with appropriate microchannel geometry.

Use of the separation well microplates enable cell culturing, cell washing, and capture of (adult) stem cells in a single separation well microplate without the need for additional plates or special wash equipment. Use of the separation well microplate also allows for multiple read outs simultaneously in a kinetic fashion.

The disclosure provides a method for co-culturing target cells, the method comprising introducing one or more target cells to a primary well of a well unit of a separation well microplate and initially incubating in a liquid media without mixing to allow the target cells to adhere to the bottom of the primary well, the primary well separated from a secondary well of the well unit of the separation well microplate by at least one closed microchannel comprising a removable barrier; introducing one or more feeder cells to the secondary well and initially incubating without mixing in a liquid media to allow the feeder cells to adhere to the bottom of the secondary well; removing the removable barrier to open the at least one closed microchannel; rocking the separation well microplate to allow mixing and media exchange between the primary well and the secondary well by gravity flow through the open microchannel; detaching target cells from the primary well; and settling the detached target cells in the primary well away from the open microchannel. The method for co-culturing target cells may be a horizontal method for co-culturing target cells. The method for co-culturing target cells may further comprise mixing the detached target cells and allowing the detached target cells to further settle in the primary well away from the open microchannel; washing the settled target cells; and collecting the washed target cells via a liquid handler.

The method for co-culturing target cells may include wherein the introducing one or more target cells to the primary well and the introducing one or more feeder cells to the secondary well are performed simultaneously or essentially simultaneously. Simultaneously may be within about 5, about 10 or about 15 minutes. Essentially simultaneously may be within about 30, about 60, or about 90 minutes.

The method for co-culturing target cells may include wherein the at least one closed microchannel is closed by a removable barrier comprising an airgap, hydrogel seal, or silicone seal, within or adjacent to the microchannel. The method for co-culturing target cells may include wherein the at least one closed microchannel is closed during at least a part of the initial incubation period.

The method for co-culturing target cells may include wherein the removable barrier comprises an airgap and the removing the removable barrier comprises eliminating the airgap after at least a part of the initial incubation period, optionally comprising using a pipette as a pump to force liquid media exchange between the primary well and the secondary well.

The method for co-culturing target cells may include wherein the detaching comprises exposing the target cell to a dissociation reagent to detach the target cells without detaching the feeder cells. The dissociation reagent may comprise a detachment enzyme, optionally in a physiological buffer. In some embodiments, the detachment enzyme may be selected from the group consisting of a trypsin, a collagenase, an elastase, a catalase, a superoxide dismutase, and a dispase. In some embodiments, the dissociation reagent comprises EDTA.

In some embodiments, the target cells may be stem cells or organoids. In some embodiments, the feeder cells are fibroblasts.

The method for co-culturing target cells may include wherein the target cells are embedded in a hydrogel dome disposed in the primary well of the well unit in the liquid media. The method for co-culturing target cells may include wherein the at least one closed microchannel is located between the bottom surface of the well unit and a bottom portion of a shared sidewall of the primary well and the secondary well.

The method for co-culturing target cells may include use of a separation well microplate comprising a plurality of well units, each well unit comprising a least one microchannel capable of fluidly connecting a primary well and a secondary well in the well unit, wherein the height of the at least one microchannel is in a range of about 10 microns to about 100 microns or about 10 microns to about 75 microns; optionally wherein the width of the microchannel is in a range of about 150 to about 500 microns, about 200 to about 400 microns, or about 300 microns. The separation well microplate may comprise a plurality of well units, for example 384 well units, 96 well units, 48 well units, 24 well units, 12 well units, 8 well units, or 6 well units.

The disclosure provides a method for quantifying secreted factors from cultivated target cells comprising kinetic measurement over time within a single separation well microplate, the method comprising cultivating target cells in a primary well of a well unit of the separation well microplate in a liquid media over an initial incubation period, the primary well separated from a secondary well of the well unit of the separation well microplate by at least one closed microchannel comprising a removable barrier, the secondary well comprising an immobilized capture reagent; removing the removable barrier to open the at least one closed microchannel to allow diffusion of the secreted factors between the primary well and the secondary well through the open microchannel after the initial incubation period; allowing the secreted factors to be captured by the immobilized capture reagent; adding labeled secondary antibodies specific for the captured secreted factors, or the immobilized capture reagent, at a first time point to form a first labeled complex; and quantifying the first labeled complex with a microplate reader.

The method for quantifying secreted factors from cultivated target cells may include wherein the immobilized capture reagent is selected from primary antibodies specific for the captured secreted factors, an avidin, and a biotin.

The method for quantifying secreted factors from cultivated target cells may further comprise adding a first antibody conjugate to the secondary well having an avidin immobilized capture reagent before removing the removable barrier, wherein the first antibody conjugate comprises secreted factor-specific primary antibodies conjugated to a biotin.

The method for quantifying secreted factors from cultivated target cells may further comprise adding a second antibody conjugate to the secondary well having a biotin immobilized capture reagent before removing the removable barrier, wherein the second antibody conjugate comprises secreted factor-specific primary antibodies conjugated to an avidin.

The method for quantifying secreted factors from cultivated target cells may further comprise adding labeled secondary antibodies specific for the captured secreted factors, the immobilized capture reagent, or the primary antibodies, at a second time point to form a second labeled complex; and quantifying the second labeled complex with a microplate reader.

The method for quantifying secreted factors from cultivated target cells may further comprise adding labeled secondary antibodies specific for the captured secreted factors, the immobilized capture reagent, or the primary antibodies at a third time point to form a third labeled complex; and detecting the third labeled complex with a microplate reader.

The method for quantifying secreted factors from cultivated target cells may include wherein the quantifying comprises multiplexed detection with imaging, homogenous with the plate reader. The method for quantifying secreted factors from cultivated target cells may include wherein the diffusion of the secreted factors comprises rocking the separation well microplate to allow diffusion of the secreted factors between the primary well and the secondary well by gravity flow through the open microchannel.

The method for quantifying secreted factors from cultivated target cells may include wherein the target cells are multicellular target cells. In some embodiments, the multicellular target cells are selected from the group consisting of organoids and tumoroids. In some embodiments, the multicellular target cells are derived from a target tissue, a biopsy sample from a patient, stem cells, tumoroid fragments, or organoid fragments.

The method for quantifying secreted factors from cultivated target cells may include wherein the cultivated organoids are embedded within a hydrogel dome in a liquid media within the primary well of the well unit.

The method for quantifying secreted factors from cultivated target cells may include wherein the removable barrier comprising an airgap, a hydrogel seal, or a silicone seal; optionally wherein the removable barrier is positioned within or adjacent to the microchannel.

The method for quantifying secreted factors from cultivated target cells may include wherein removing of the removable barrier comprises eliminating the airgap; optionally comprising using a pipette as a pump to force liquid media exchange between the primary well and the secondary well.

The method for quantifying secreted factors from cultivated target cells may include wherein the immobilized capture reagent is immobilized at the bottom of the secondary well. In some embodiments, the immobilized capture reagent comprises a plurality of primary antibodies specific for a plurality of secreted factors, wherein the primary antibodies are arranged in the bottom of the secondary well in a detection array. The method for quantifying secreted factors from cultivated target cells may include wherein the secreted factors are selected from the group consisting of hormones, antibodies, cytokines, chemokines, and growth factors.

The disclosure provides a method of isolating a single cell type from a cultivated multicellular target cell culture within a single separation well microplate, the method comprising cultivating multicellular target cells in a primary well of a well unit of the separation well microplate in a liquid media over an initial incubation period, the primary well separated from a secondary well of the well unit of the separation well microplate by at least one closed microchannel comprising a removable barrier, the secondary well comprising a surface functionalized with an immobilized capture reagent; dissociating the multicellular target cells in the primary well to form a mixture of individual cells comprising the single cell type and cells of no interest; removing the removable barrier to open the microchannel to allow the mixture of individual cells to travel through the open microchannel to the secondary well and allowing the single cell type associate with the immobilized capture reagent in the secondary well to form an immobilized complex; washing the primary well and the secondary well to remove the unbound cells of no interest; and isolating the single cell type comprising dissociating the immobilized complex. The allowing individual cells to travel through the open microchannel may comprise using a pipette as a pump, diffusion, or laminar flow with appropriate microchannel geometry.

The method of isolating a single cell type from a cultivated multicellular target cell culture may further comprise reseeding the isolated single cell type in a primary well of the separation well microplate. In some embodiments, the single cell type is a stem cell. In some embodiments, the single cell type is a target cell.

In some embodiments, the multicellular target cells are organoids.

The method of isolating a single cell type from a cultivated multicellular target cell culture may include wherein the immobilized capture reagent is disposed at the bottom of the secondary well or on a scaffold within the secondary well.

In some embodiments, the immobilized capture reagent may be selected from primary antibodies specific for a surface marker of the single cell type, an avidin, and a biotin.

The method of isolating a single cell type from a cultivated multicellular target cell culture may further comprise adding a first antibody conjugate to the secondary well having an avidin immobilized capture reagent before removing the removable barrier, wherein the first antibody conjugate comprises a single cell type surface marker-specific primary antibody conjugated to a biotin.

The method of isolating a single cell type from a cultivated multicellular target cell culture may further comprise adding a second antibody conjugate to the secondary well having a biotin immobilized capture reagent before removing the removable barrier, wherein the second antibody conjugate comprises a single cell type surface marker-specific primary antibody conjugated to an avidin.

The method of isolating a single cell type from a cultivated multicellular target cell culture may include wherein the multicellular target cells are cultivated in a hydrogel dome in the liquid media within the primary well.

The disclosure provides a method of isolating a stem cell from a multicellular target cell culture within a single separation well microplate, the method comprising cultivating multicellular target cells in a first hydrogel dome in a liquid media in primary well of a well unit of the separation well microplate over an initial incubation period, the primary well separated from a secondary well of the well unit of the separation well microplate by at least one closed microchannel comprising a removable barrier; dissociating the multicellular target cells in the primary well to form a mixture of individual cells comprising the stem cells and cells of no interest; adding magnetic beads comprising an immobilized capture reagent specific for the stem cells to the primary well and further incubating to allow binding of the magnetic beads to the stem cells; applying a magnet underneath the primary well to retain the magnetic beads-stem cell complex; removing the removable barrier to open the microchannel and washing the retained magnetic bead-stem cell complex to remove the unbound cells of no interest by aspirating liquid media through the secondary well; adding a second hydrogel dome to the washed retained magnetic bead-stem cell complex in the primary well; removing the magnet from underneath the primary well; dissociating the stem cells from the magnetic beads; and applying a magnet above the primary well to levitate the magnetic beads and isolate the stem cells in the second hydrogel dome.

In some embodiments, the method of isolating a stem cell from a multicellular target cell culture further comprises removing the magnet from underneath the primary well; transferring the washed retained magnetic bead-stem cell complex to a new primary well in a new well unit of a new separation well microplate; and reapplying a magnet underneath the new primary well in the new well unit of the new separation well microplate. In some embodiments, the immobilized capture reagent is selected from primary antibodies specific for a surface marker of the stem cells, an avidin, and a biotin.

The disclosure provides a method for selecting a therapy for cancer treatment of a subject in need thereof, the method comprising cultivating multicellular target cells derived from the subject in a primary well of a well unit of a separation well microplate in a liquid media over an initial incubation period, the primary well separated from a secondary well of the well unit of the separation well microplate by at least one closed microchannel comprising a removable barrier, the secondary well comprising a candidate cancer therapeutic immune cell, monoclonal antibody, immune system modulator, or antineoplastic agent; removing the removable barrier to open the at least one closed microchannel to allow diffusion between the primary well and the secondary well through the open microchannel over a second incubation period; imaging the secondary well and optionally the primary well after the second incubation period to quantify cell migration or multicellular target cell growth; and selecting the candidate therapeutic based on increased immune cell migration within the secondary well or between the secondary well and the primary well compared to a control well unit without multicellular target cells; or decreased multicellular target cell growth in the primary well compared to a control well unit without candidate therapeutic.

The method for selecting a therapy for cancer treatment may comprise wherein the multicellular target cells are embedded in a hydrogel dome disposed in the primary well of the well unit in the liquid media. The method for selecting a therapy for cancer treatment may comprise wherein the multicellular target cells are derived from a biopsy sample from the subject or a tumoroid fragment. In some embodiments, the immune cells may be CAR T-cells.

The disclosure provides an automated method of breaking a hydrogel dome, the method comprising cultivating a first cell in a hydrogel dome in a liquid media in a well of a microplate over an initial incubation period of time to provide multicellular target cells; puncturing the hydrogel dome at or near the x,y center position of the dome in the well with a pipette tip associated with a liquid handler after the initial incubation period of time; moving the pipette tip through the dome in a multiplicity of steps, each step comprising an X,Y predetermined position within the well to break the hydrogel dome and optionally liquify the hydrogel dome to free the target cell from the hydrogel dome within the well. The pipette may be aspirated at one or more, two or more, a multiplicity of, or each of the predetermined positions.

The disclosure provides an automated method of breaking a hydrogel dome and isolating a target cell, the method comprising cultivating a first cell in a hydrogel dome in a liquid media in a well of a microplate over an initial incubation period of time to provide multicellular target cells; puncturing the hydrogel dome at or near the x,y center position of the dome in the well with a pipette tip associated with a liquid handler after the initial incubation period of time; moving the pipette tip through the dome in a multiplicity of steps to free the target cells from the hydrogel dome, each step comprising an X,Y predetermined position within the well to break the hydrogel vdome and optionally liquify the hydrogel dome; and isolating one or more of the target cells from the well. The pipette may be aspirated at one or more, two or more, or each of the predetermined positions.

Each of the X, Y predetermined positions within the well may be calculated comprising:
X(stepnumber)=Xwellcenter+alpha/steps*stepnumber*COS(theta/steps*stepnumber); and
Y(stepnumber)=Ywellcenter+alpha/steps*stepnumber*SIN(theta/steps*stepnumber), wherein alpha=dome diameter (mm)/1.8; steps=number of steps for moving the pipette tip through the hydrogel dome; Xwellcenter=x position of dome center within the well; Ywellcenter=y position of dome center within the well; and theta=a constant value between 5 and 50. In some cases, the number of steps is an integer from 2-20, 3-18, 4-17, 5-15, 6-12, or 8-10. In some cases, theta is a constant from 5-50, 10-45, 15-40, 15-35, or any number in between. In some cases theta is selected from the group consisting of 5, 10, 12, 13, 14, 15, 15.7, 16, 17, 18, 20, 22, 24, 26, 28, 30, 32, 34, 36, 38, 40, 45, or 50, or any number in between.

In some cases, the liquid handler aspirates the pipette tip within the hydrogel dome and/or the pipette tip punctures the hydrogel dome at one or more, two or more, a multiplicity of, or each of the predetermined positions.

In some cases, the well is a primary well of a well unit of a separation well microplate.

The x,y center position of the hydrogel dome may be determined by one or more parameters selected from the group consisting of the original seeding position in the well, volume of hydrogel deposited in the well, and imaging of the hydrogel dome in the well.

The diameter of the hydrogel dome may be determined by the volume of the hydrogel deposited in the well and/or imaging of the hydrogel dome within the well.

The X,Y predetermined positions may form a pattern in the well selected from the group consisting of a spiral pattern, a star pattern, and a zig-zag pattern within and/or through the hydrogel dome.

The pipette tip may be dragged beneath the surface of the hydrogel dome between each of the positions. The pipette tip may be lifted above the surface of the hydrogel dome between each of the positions.

The target cells may be multicellular target cells, optionally selected from the group of spheroids, tumoroids, and organoids.

In some cases, the method further comprises dissociating the multicellular target cells from each other to provide individual target cells prior to the isolating.

### BRIEF DESCRIPTION OF THE DRAWINGS

FIG. 1A shows a first view of an exemplary single well unit 100 in a separation well microplate useful for horizontal co-culture for cell/organoid feeding. A primary well 112 is separated from a secondary well 115 by at least one microchannel 118. In some embodiments, the at least one microchannel 118 may be open to allow for media exchange between primary well 112 and secondary well 115. Alternatively, the at least one microchannel 118 may comprise a removable barrier to isolate primary and secondary wells from each other.

FIG. 1B shows a second view of an exemplary single well unit 100 in a separation well microplate useful for horizontal co-culture for cell/organoid feeding. A primary well 112 is separated from a secondary well 115 by at least one microchannel 118. Media exchange occurs by gravity flow through the microchannel 118 while the separation well microplate sits on a rocker.

FIG. 1C shows an example of a cross section of a single well unit 100 of a separation well microplate associated with the cell culturing methods according to various embodiments of the present disclosure. A primary well 112 is separated from a secondary well 115 by a shared sidewall comprising at least one microchannel 118 between the primary well 112 and the secondary well 115. The primary well 112 may contain target cells (e.g., organoids) embedded in a hydrogel dome 106. The primary well 112 and the secondary well 115 may be at least partially filled with a liquid media 109. The bottom of the well unit 100 comprises a bottom layer sheet 121 comprising a viewing window that is optically transparent to allow for imaging.

FIG. 2 shows a representative schematic of an exemplary method for detecting multiple secreted factors from target cells (e.g., organoids) allowing cell cultivation and kinetic measurement of secreted factors within a single separation well microplate. The target cells (e.g., organoids) are cultivated in the primary well 112. The target cells generate secreted factors (e.g., hormones) that are allowed to diffuse through the microchannel 118. The secreted factors are captured in the secondary well 115 comprising an array of coated/surface functionalized immobilized antibodies specific for the secreted factors.

FIG. 3 shows an exemplary method for performing single cell isolation (e.g., stem cells) from multicellular target cell culture (e.g., organoids) within a single separation well microplate comprising a surface functionalized with an immobilized capture reagent (e.g., stem cell-specific antibodies, an avidin, or a biotin). Multicellular target cells (e.g., organoids) are cultivated in a hydrogel dome 106 in a liquid media 109 within the primary well 112, the secondary well 115 is coated/surface functionalized by immobilized antibodies specific for the cells of interest (e.g., stem cells), and the microchannel 118 is closed by a removable barrier (310). After a period of time, the multicellular target cells (e.g., organoids) are dissociated to form a mixture of individual cells including the cells of interest (320). The barrier is removed from the microchannel 118, and the mixture of individual cells is able to travel through the open microchannel 118, for example, hydraulically by using a pipette as a pump, or by laminar flow with appropriate microchannel geometry. The cells of interest are captured by the immobilized specific antibodies in the secondary well 115, and cells of no interest are washed out of the microwell (330). Cells of interest (e.g., stem cells) are dissociated from the antibody/cell complex (340). Cells of interest (e.g., stem cells) may be used to reseed a new well (350).

FIG. 4 shows an exemplary method for performing stem cell isolation from organoid culture within a single separation well microplate comprising an antibody coated scaffold. Multicellular target cells (e.g., organoids) are cultivated in a hydrogel dome 106 in a liquid media 109 within the primary well 112, the secondary well 115 comprises a scaffold containing coated/surface functionalized immobilized antibodies specific for the cells of interest (e.g., stem cells), and the microchannel 118 is closed by a removable barrier (410). After a period of time, the multicellular target cells (e.g., organoids) are dissociated to form a mixture of individual cells including the cells of interest (420). The barrier is removed from the microchannel 118, and the mixture of individual cells is able to traverse the open microchannel 118, for example, hydraulically by using a pipette as a pump, or by laminar flow with appropriate microchannel geometry. The cells of interest are captured by the scaffold comprising immobilized specific antibodies in the secondary well 115, and cells of no interest are washed out of the microwell (430). Cells of interest (e.g., stem cells) are dissociated from the antibody/cell complex in the scaffold (440). Cells of interest (e.g., stem cells) may be used to reseed a new well (450).

FIG. 5 shows an exemplary method for passaging in a way to break up multicellular target cells (e.g., organoids) to the single cell level with isolation of desired single cells (e.g., stem cells) using magnetic beads coated with a capture reagent (e.g., cell specific antibodies or biotin) in a single separation cell microplate. Multicellular target cells (e.g., organoids) are cultivated in a well unit of a separation well microplate comprising a primary well 112 containing the organoids 103 (e.g., intestinal organoids) within a hydrogel dome 106 in a liquid media 109 disposed at the bottom surface of the primary well 112, with a closed microchannel 118 comprising a removable barrier between primary and secondary wells. After a period of time, the multicellular target cells (e.g., organoids) are dissociated to form a mixture of individual cells including the cells of interest (520). Magnetic beads surface functionalized with an immobilized capture reagent (e.g., an antibody specific for stem cell markers or a biotin) are added to the mixture of individual cells in the primary well unit (530). The magnetic beads are allowed to incubate with the mixture of individual cells to allow binding of the desired single cells (e.g., stem cells) (540). A magnet is applied to the bottom of the primary well to retain the magnetic bead-capture reagent-stem cell complex, the microchannel 118 is opened by removing the barrier, and the unbound cells and debris are washed through the secondary well 115 by, for example, aspirating the liquid media with a liquid handler. A new hydrogel dome may be formed in the primary well such that the stem cells and magnetic beads are embedded in the hydrogel (560), and the magnet is removed from under the primary well (570). A magnet may be placed at the top of the primary well to levitate the stem cells in the hydrogel (580).

FIG. 6A shows an exemplary method for evaluating cell (e.g., lymphocyte) functionality and toxicology studies in a single separation well microplate. Multicellular target cells (e.g., tumoroids, organoids) are cultivated in a hydrogel dome 106 in a liquid media 109 in a primary well 112 of a well unit 100 of a separation well microplate. The secondary well 115 comprises an immune cell (e.g., CAR-T cells, CAR-NK cells, other lymphocytes). A chemoattractant may be emitted by the multicellular target cells and diffuse through the open microchannel 118 which may cause certain immune cells to migrate within the secondary well 115 or even into the primary well 112, depending on microchannel 118 configuration. The microchannel 118 may have a channel size of 5 microns while the lymphocytes have a size of 7-10 microns. In some embodiments, the multicellular target cells may emit a chemoattractant which may cause immune cell migration into the primary well 112, depending on microchannel 118 configuration.

FIG. 6B shows an exemplary method for evaluating cell (e.g., lymphocyte) functionality and toxicology studies in a single separation well microplate wherein a tilted position may be employed to impair immune cell migration. Multicellular target cells (e.g., tumoroids, organoids) are cultivated in a hydrogel dome 106 in a liquid media 109 in a primary well 112 of a well unit 100 of a separation well microplate. The secondary well 115 comprises an immune cell (e.g., CAR-T cells, CAR-NK cells, other lymphocytes). A chemoattractant may be emitted by the multicellular target cells and diffuse through the open microchannel 118 which may cause certain immune cells to migrate within the secondary well 115 or even into the primary well 112, depending on microchannel 118 configuration. The separation well microplate may be held in a tilted position to impair immune cell migration to the primary well 112.

FIG. 7A shows exemplary spreadsheet calculations for hydrogel dome breaking pattern of X,Y predetermined positions of automated pipettor movements calculated using formulae:
X(stepnumber)=Xwellcenter+alpha/steps*stepnumber*COS (theta/steps*stepnumber); and
Y(stepnumber)=Ywellcenter+alpha/steps*stepnumber*SIN (theta/steps*stepnumber), wherein dome diameter (mm)=8, number of steps=9, alpha=diameter/1.8, and theta=15.7. A spiral pattern of steps is formed, as shown in FIG. 7B (lower right panel).

FIG. 7B shows exemplary pipettor movement patterns of predetermined positions calculated for hydrogel dome breaking using preceding formulae with 6 steps (upper left panel), 7 steps (upper right panel), 8 steps (lower left panel), and 9 steps (lower right panel). When using theta value of 15.7, a spiral pattern of steps is formed for each pattern.

FIG. 7C shows exemplary pipettor movement patterns of predetermined positions calculated for hydrogel dome breaking using preceding formulae with 10 steps (upper left panel), 11 steps (upper right panel), 12 steps (lower left panel), and 13 steps (lower right panel). When using theta value of 15.7, a spiral pattern of steps is formed for each pattern.

FIG. 7D shows exemplary pipettor movement patterns calculated for hydrogel dome breaking using preceding formulae with wherein diameter (mm)=8, number of steps=13, alpha=diameter/1.8, and theta=30. When using theta value of 30, the pattern of steps appears less like a spiral and a star-like pattern begins to emerge.

### DETAILED DESCRIPTION

Studying co-culture and cell interaction contains multiple hurdles for culture and data analysis. Separation of different cell types in separate compartments can allow efficient cross-talk and interaction of cell types and tissues, including but not limited to cell migration, neurite growth, angiogenesis, immune cell migration and killing, metastasis, cell invasion, monocyte adhesion, apoptosis, cell differentiation, stem cell implantation, inflammation, and secretion of multiple growth factors and metabolites.

Methods are provided for cell/organoid co-culture assays comprising use of separation well microplates. The separation well microplates support horizontal co-culturing in an automation-friendly fashion.

Methods are provided for co-culturing different cell types in adjacent wells that can be hydraulically connected using a pipette as a pump, or connected by laminar flow via microchannel between primary well and secondary well.

In support of the present disclosure, methods of culturing organoids and cells using separation well microplates were investigated. For example, organoids were dissociated and passed to new wells. Long term organoid culture exhibited good status of the cultured cells in the passaging plate.

Adhesion of fibroblasts was evaluated on plasma treated separation well microplates. Adhesion and normal behavior of fibroblasts was observed. Migration of fibroblasts through the channel was tested using a chemoattractant.

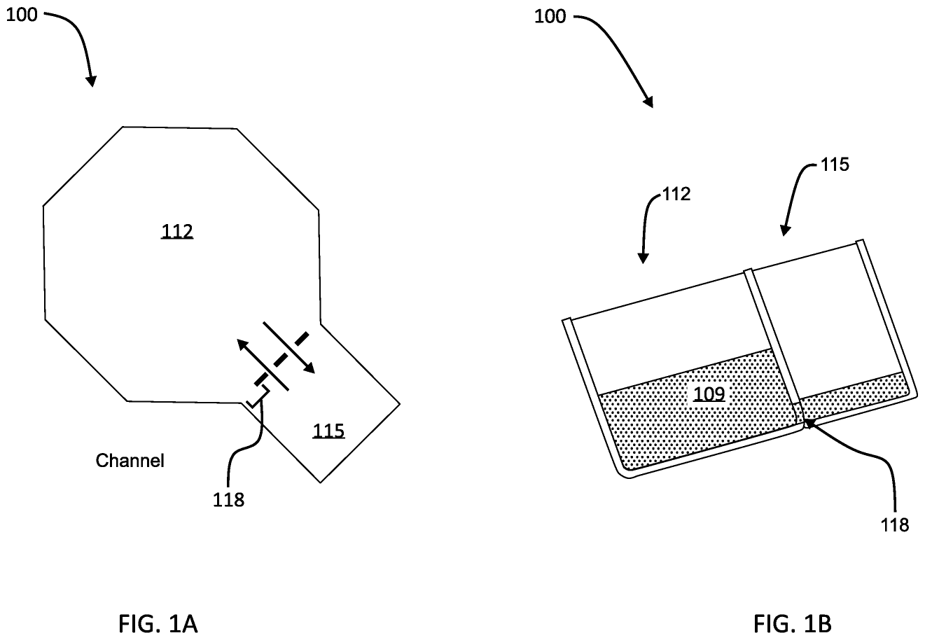

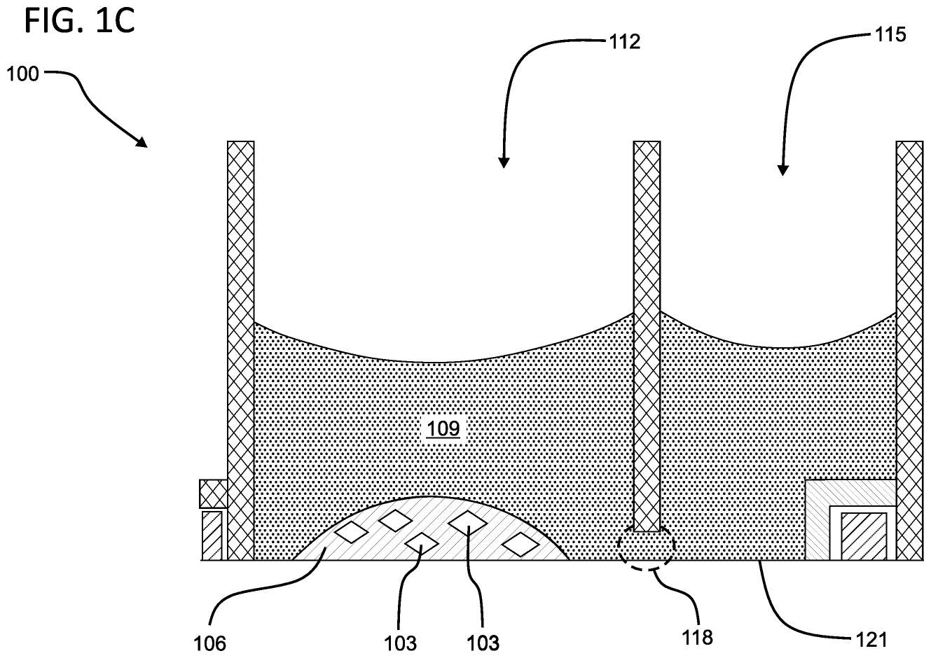

Methods of the disclosure employ a separation well microplate having a well geometry including a primary well and a secondary chamber connected by at least one channel. For example, the culturing methods of the present disclosure may utilize separation well microplates having a multiplicity of well units each including a primary well and a secondary chamber connected by at least one channel. For example, each well unit may comprise a well geometry as shown in FIG. 1 comprising primary well that is fluidly connected to a secondary well via at least one channel. Separation well microplates are described in U.S. Provisional Application 63/151,082 entitled “METHODS AND APPARATUSES FOR CELL PASSAGING USING MICROPLATE WELL UNITS” filed on Feb. 19, 2021, U.S. patent application Ser. No. 17/580,193 entitled “MICROPLATES FOR AUTOMATING ORGANOID CULTIVATION” filed on Oct. 22, 2021, U.S. Provisional Application 63/300,294 entitled “3D PRINTING OF ORGANOID PASSAGING PLATE” filed on Jan. 18, 2022, and PCT International Application PCT/IB2021/062356, entitled “MICROPLATE WELLS FOR CELL CULTIVATION” filed on Dec. 27, 2021, each of which is incorporated by reference herein in its entirety.

FIG. 1A illustrates one example of a well unit 100 of a separation well microplate that can be used in connection with the methods of the present disclosure. In particular, FIG. 1A illustrates an example view of a well unit 100 of a microplate that can be used for growing, culturing, monitoring, and assaying embryoid bodies, fused embryoid bodies, spheroids, organoids, or other multi-cellular bodies in accordance with various embodiments of the present disclosure. For example, the single well unit 100 of a separation well microplate is useful for horizontal co-culture for cell/organoid feeding. A separation well microplate for use in methods according to the disclosure may comprise a plurality of well units, each well unit comprising a primary well 112 that is separated from a secondary well 115 by a shared sidewall, and primary and secondary wells may be fluidly connected by at least one microchannel 118. In some embodiments, the at least one microchannel 118 may comprise a removable barrier to isolate primary and secondary wells from each other. In some embodiments, the at least one microchannel 118 may be open to allow for media exchange between primary well 112 and secondary well 115. The well unit 100 of the separation well microplate includes a primary well 112 which may be fluidly connected to a secondary well 115 by at least one microchannel 118. In some examples, the primary well 112 may be used as a cultivation well and the secondary well 115 may be used as a feeding well. The microchannel 118 may contain a removable barrier. The at least one microchannel 118 may be closed by a removable barrier or may be open. The one or more microchannels 118 between the primary well 112 and secondary well 115 may be closed by a removable barrier, for example, by an air gap, hydrogel seal, or silicone seal. The one or more microchannels 118 between the primary well 112 and secondary well 115 may be closed, for example, by an air gap. The air gap may be formed by, for example, air bubbles.

FIG. 1B illustrates another example well unit 100 of a separation well microplate that can be used in connection with the methods of the present disclosure. In FIG. 1B, the well unit 100 includes a primary well 112 and secondary well 115 that can be fluidly connected by an open at least one microchannel 118. When open, the at least one channel 118 geometry may allow for exchange of media, expressed proteins such as hormones, nutrients, growth factors, waste products, and debris between primary well and secondary wells, while retaining, for example, target cells such as organoids in primary wells 112 and retaining feeder cells in secondary wells 115. The dimensions of the at least one microchannel 118 may prevent the passage of multicellular target cells (e.g., organoids, tumoroids, etc.) through the microchannel 118. In some examples, the at least one microchannel 118 geometry may include a slit configuration having a channel width of 150-500 microns or more, or 200-400 microns, or about 300 microns×10-100 microns, or 10-75 microns high. During the methods of the disclosure, the at least one microchannel 118 between primary and secondary wells may be closed by a removable barrier or may be opened to allow for exchange of media, secreted factors, hormones, growth factors, nutrients, waste products and debris between primary well 112 and secondary well 115, for example, by gravity feed while rocking the separation well microplate. In some examples, the at least one microchannel may be opened by, for example, removing a removable barrier such as eliminating the airgap by using a pipette as a pump, for example, sealing a pipette tip to the secondary well to force liquid flow between the two wells.

FIG. 1C illustrates a further example well unit 100 of a separation well microplate that may be used in connection with the methods of the present disclosure. The well unit 100 includes a primary well section 112, a secondary well section 115, at least one microchannel 118, and a first hydrogel dome 106 that may be disposed in the primary well section 112 of the well unit 100. According to various embodiments, the well unit 100 further comprises a bottom layer sheet 121 disposed on an underside of a well plate body of the microplate. The bottom layer sheet 121 is attached to the underside of the well platebody forming the bottom surface of the well unit 100. In various examples, the bottom layer sheet 121 comprises a viewing window that is optically transparent to allow forimaging of spheroids, organoids, or other cell cultures being cultured in the well unit100 of the microplate, as can be appreciated. The viewing window can be a window that is suitable for microscopic observation, whether brightfield, phase-contrast, fluorescent, confocal, two-photon, or other microscopic imaging modalities as known in the art. In some examples, the bottom layer sheet 121 may comprise a gas permeable sheet that is configured to increase an oxygen supply for the growing spheroids, organoids, or other cellular bodies in the well unit 100 of the microplate. The gas permeable sheet can be formed of a material comprising polytetrafluoroethylene (PTFE), PEFP, Polyimide, Polydimethylsiloxane (PDMS), Polycarbonate, and/or other material as can be appreciated. According to various examples, the gas permeable sheet can have a thickness of about 5-70 microns. According to various examples, the gas permeable sheet can comprise a plurality ofpores. In other examples, the gas permeable sheet can allow molecules to pass by diffusion. Alternatively, the gas permeable sheet can comprise some other thickness, pore diameter, and pore density.

As shown in FIG. 1C, target cells in the form of multicellular bodies 103 (e.g., spheroids, tumoroids, organoids, etc.) may be embedded in a dome of hydrogel 106 (e.g., Matrigel®) that is disposed in the well unit 100 in the primary well 112 and is surrounded by a liquid medium 109. According to various embodiments, the liquid medium 109 can contain the appropriate growth factors and supplements to create and/or stimulate growth of the desired multi-cellular body. Exemplary growth factors that can be suitable include angiopoietin, bone morphogenetic proteins (BMPs), ciliary neurotropic factor, colony stimulating factors, ephrins, epidermal growth factor, erythropoietin, fibroblast growth factors, glial-derived neurotrophic factor, hepatocyte growth factor, insulin, insulin-like growth factors, interleukins, leukemia inhibitory factor, keratinocyte growth factor, neuregulins, neurotrophins, platelet-derived growth factor, transforming growth factors, tumor necrosis factor (alpha), vascular endothelial growth factor, and/or the like.

According to various embodiments, the well unit 100 of FIG. 1C comprises a primary well section 112 and a secondary well section 115. In various examples, the primary well section 112 and the secondary well section 115 are isolated from one another by a closed microchannel. In some embodiments, the microchannel 118 may be closed by, for example, an airgap, hydrogel, or a silicone. In some embodiments, the microchannel 118 may be open such that the primary well section 112 and the secondary well section 115 are in fluid connection with one another via at least one microchannel 118, for example, to facilitate a gravitational flow of liquid (e.g., liquid medium 109) between the primary well section 112 and the secondary well section 115 in response to a tilting of the microplate. Exchanging the liquid medium 109 between the primary well section 112 and the secondary well section 115 may allow for removal of toxic by-products and may supply the growing cell cultures with fresh nutrients. In the example of FIG. 1C, the hydrogel 106 and the cellular bodies 103 are disposed in the primary well section 112 of the well unit 100.

According to various embodiments, the methods according to the present disclosure employ a separation well microplate comprising a multiplicity of well units 100, such as, for example, 384 well units, 96 well units, 48 well units, 24 well units, 12 well units, 8 well units, or 6 well units. Dependent upon the number of well units 100 in the microplate, the width of the primary well section 112 can be up to about 8 millimeters (mm) (e.g., for 96 well plate), up to 11 mm (e.g., for a 48 well plate), up to about 17 mm (e.g., for a 24 well plate), and/or other sizes as can be appreciated. In addition, the depth of the primary well section 112 and the secondary well section 115 is specified such that the microplate can be tilted to allow fluid exchange within the well units 100 without spilling the fluid out of the respective primary well section 112 or secondary well section 115 of each the well units 100.

According to various embodiments, the well unit 100 of the separation well microplate comprises a primary well section 112 that may be sized and shaped to support deposited cellular bodies 103 (e.g., cell aggregates) that can be embedded in a hydrogel 106 that is deposited into the primary well section 112. For example, the primary well section 112 can be considered a culture well that is used to grow target cells such as, for example, embryoid bodies, fused embryoid bodies, spheroids, organoids, and/or other multi-cellular bodies.

The secondary well section 115 can be used to grow feeder cells, supply feeding media and/or other nutrients, that can be used to feed the growing cell aggregates positioned in the primary well section 112. In some embodiments, the secondary well may be coated with primary antibodies specific for secreted factors, for example, from the target cells. In some embodiments, the secondary well section 115 can be used to harvest supernatant from the cell aggregates. For example, the secondary well section 115 can be considered a supply well that comprises the feeding media and/or other nutrients that can be used by the growingcell culture in the primary well section 112. The secondary well section 115 may be sizedand shaped to hold fluid that can be exchanged with the primary well section 112 according to various embodiments of the present disclosure.

According to various embodiments and dependent upon a number of well units 100 in the microplate, thewidth of the secondary well section 115 can be up to about 8 millimeters (mm) (e.g., for 96 well plate), up to 11 mm (e.g., for a 48 well plate), up to about 17 mm (e.g., fora 24 well plate), and/or other sizes as can be appreciated.

According to various embodiments, the size and shape of the primary well section 112 and the secondary well section 115 can differ from one another. In some examples, the primary well section 112 is larger (in a dimension, for example diameter, cross-section, or volume) than the secondary well section 115. In other examples, the secondary well section 115 is larger than the primary well section 112. In some examples, the primary well section 112 comprises a shape that differs from a shape of the secondary well section 115.

According to various examples, the well unit of the separation well microplate comprises at least one microchannel 118 that is sized and shaped to prevent objects having dimensions (e.g., diameter, height, width, etc.) of a certain size (e.g., greater than about twenty-five (25) microns (μ)) from passing from one well section to the other well section.

In some examples, the height of the at least one microchannel 118 can be sized between about ten (10) microns up to about one hundred (100) microns, or about 10 microns to about 75 microns, or about ten (10) microns to about twenty-five (25) microns. The at least one microchannel 118 geometry may include a slit configuration having a channel width of 150-500 microns or more, or 200-400 microns, or about 300 microns×10-100 microns, or 10-75 microns high. For example, the height of the at least one microchannel 118 may be such to prevent objects (e.g., spheroids, tumoroids, organoids, organoid fragments, etc.) that are sized at about 25 μor greater that are present in the primary well section 112 will be prevented from migrating to secondary well section 115. The microchannel 118 can be closed by, for example, an airgap, a hydrogel seal, or a silicone seal. The microchannel 118 can be opened by, for example, eliminating the airgap or removing the hydrogel seal or silicone seal.

In some embodiments of the present disclosure, the primary well 112 may be utilized as a cultivation chamber. Initially, the at least one channel 118 may be closed, for example, target cells may be cultivated and allowed to attach to the bottom of the primary wells 112 of the separation well microplate, for example, without mixing. Feeder cells may be cultivated and allowed to attach to the bottom of secondary wells 115 of the separation well microplate, for example, without mixing. After adherence of target cells and/or feeder cells to wells, the closed at least one channel 118 may be opened. The separation well microplate may be placed on a rocker to allow exchange of media, hormones, growth factors, nutrients, waste products, and debris by gravity feed. In some examples, feeder cells may be cultivated in the secondary chamber 115, allowing target cells cultivated in the primary chamber 112 to be fed through the open at least one channel.

### Definitions

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” refers to and encompasses any and all possible combinations of one or more of the associated listed items.

The term “about,” when referring to a measurable value such as an amount of a compound, dose, time, temperature, and the like, is meant to encompass variations of 10%, 5%, 1%, 0.5%, or even 0.1% of the specified amount.

The term “antibody conjugate” refers to a specific antibody conjugated to a chromophore or fluorophore molecule, an avidin such as a streptavidin, or a biotin, wherein the antibody is specific for a target antigen such as a secreted factor.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

“Treating” or “treatment” of a disease state or condition includes: (i) preventing the disease state or condition, i.e., causing the clinical symptoms of the disease state or condition not to develop in a subject that may be exposed to or predisposed to the disease state or condition, but does not yet experience or display symptoms of the disease state or condition, (ii) inhibiting the disease state or condition, i.e., arresting the development of the disease state or condition or its clinical symptoms, or (iii) relieving the disease state or condition, i.e., causing temporary or permanent regression of the disease state or condition or its clinical symptoms.

The term “feeder cells” refers to cells which provide extracellular secretions to help another cell to proliferate, grow, differentiate, and/or maintain identity. Feeder cells may support growth of target cells in culture by contributing a complex mixture of extracellular matrix (ECM) components and growth factors. In some cases, the feeder cells may be unable to divide, i.e., have arrested cell growth. Feeder cell growth may be arrested by, for example, any appropriate methods known in the art. Feeder cell growth may be arrested by chemical fixation, for example, by mitomycin-C or glutaraldehyde chemical fixation. Feeder cell growth may be arrested by physical methods, for example, gamma irradiation, x-ray irradiation, or electric pulses. For example, feeder cells used for co-culture of target cells such as embryonic stem cells (ESCs) may be fibroblasts which may be mitotically inactivated so they remain viable. In some cases, target cells may be grown in the presence of feeder cells capable of dividing. Some live feeder cells (such as human fibroblasts) may also become target cells as in the case of induced pluripotent stem cells (iPSCs) upon reprogramming. Feeder cells may be arrested feeder cells, for example, that are unable to divide. Feeder cell selection may be dependent on target cells. Feeder cells may be, for example, fibroblasts that are not arrested. Feeder cells may be arrested fibroblasts, epithelial cells, mesenchymal cells, muscle cells, stromal cells, spleen cells, or amniocytes. The fibroblasts may be, for example, human dermal fibroblasts, 3T3 fibroblast cells, human fetal fibroblasts, mouse embryonic fibroblasts, and the like. The epithelial cells may be, for example, human adult fallopian tubal epithelial cells, human amniotic epithelial cells, HeLa cells (human cervical cancer carcinoma epithelial cells), and the like. The mesenchymal cells may be adipose-derived mesenchymal stem cells, human bone marrow-derived mesenchymal stem cells, human bone marrow-derived mesenchymal cells, human amniotic mesenchymal stem cells, and the like. The stromal cells may be, for example, human bone marrow stromal cells, or mouse bone marrow stromal cells. The amniocytes may be, for example, human amniocytes or mouse amniocytes.

The term “target cells” refers to cells for automated cell culture applications of the present disclosure, such as organoids, tumoroids, spheroids, stem cells, or a production cell line. In some embodiments, the target cells are spheroids, tumoroids, organoids and/or other multi-cellular bodies. In some embodiments, the target cells may be stem cells. In some embodiments, the target cells may be a production cell line. The target cells may be derived from a target tissue. The target tissue may be a mammalian primary tissue, or an organoid, or tumoroid. The mammalian tissue may be derived from a patient biopsy sample. The target tissue may be derived from target organs such as, e.g., lung, intestine such as small intestine, colon, stomach, pancreas, liver, kidney, skin, bone marrow, blood-brain barrier, brain, heart, and the like.

Organoid, spheroid, tumoroid, and three-dimensional (3D) cell culture models are useful in many applications such as disease modeling and regenerative medicine. 3D cellular models like organoids and spheroids may be useful to better understand complex biology in a physiologically relevant context because cells often retain natural shape and proper spatial orientation, such as in aggregates or spheroids, whereas 2D models of cells grown in a sheet or monolayer may not be as successful. Gene and protein expression of 3D cell culture may more closely mimic gene and protein expression. For example, 3D cell cultures may be useful for drug target identification, lead compound identification, compound optimization, preclinical attesting, solid tumor modeling, genetic disease modeling, drug discovery, precision medicine, organs-on-chips, and bioprinting.

The term “spheroids” refer to three dimensional (3D) multicellular in vitro tissue cultures aggregates composed of one or more cells types that grow and proliferate, and may exhibit enhance physiological responses, but do not undergo differentiation or self-organization. Common cell sources for spheroids are primary tissues or immortalized cell lines. Spheroids may bridge the gap between monolayers and complex organs.

The term “organoids” refer to three dimensional (3D) multicellular in vitro tissue culture aggregates composed of one or more cell types, in which cells spontaneously self-organize into properly differentiated functional cell types and progenitors that resemble their in vivo counterparts in at least one aspect. Organoids mimic their corresponding in vivo organs. Organoids can be derived from pluripotent stem cells (PSCs), induced pluripotent stem cells (iPSCs), neonatal tissue stem cells, embryonic stem cells (ESCs), adult stem cells, or primary tissue. Organoid cultures can be crafted to resemble much of the complexity of an organ, therefore are useful for study of disease etiology and treatment. Organoid technology has recently emerged as an essential tool for both fundamental and biomedical research. The organoid cultures may be selected from different types of target organs such as, e.g., lung, intestine such as small intestine, colon, stomach, pancreas, liver, kidney, skin, bone marrow, blood-brain barrier, brain, heart, and the like.

For example, stomach organoid tissue cultures may be derived from a source such as, e.g., adult mouse, adult human, hPSC, and the like. Stomach organoid tissue cultures may employ a stem cell culture condition (niche factors) including media components such as one or more of EGF, Noggin, R-spondin, Wnt-3A, FGF10, and the like, depending on source. The differentiation culture condition may include EGF, R-spondin EGF, R-spondin, and the like, depending on source.

As another example, small intestine organoid tissue cultures may be derived from a source such as, e.g., adult mouse, adult human, hPSC, and the like. Small intestine organoid tissue cultures may employ a stem cell culture condition (niche factors) including media components such as one or more of EGF, Noggin, R-spondin, Wnt-3A TGF-beta inhibitor, p38 inhibitor, and the like, depending on source. The differentiation culture condition may include EGF, Noggin, TGF-beta inhibitor and the like, depending on source.

As a further example, colon organoid tissue cultures may be derived from a source such as, e.g., adult mouse, adult human, and the like. Colon organoid tissue cultures may employ a stem cell culture condition (niche factors) including media components such as one or more of EGF, Noggin, R-spondin, Wnt-3A TGF-beta inhibitor, p38 inhibitor, and the like, depending on source. The differentiation culture condition may include EGF, Noggin, TGF-beta inhibitor and the like, depending on source.

As another example, pancreas organoid tissue cultures may be derived from a source such as, e.g., adult mouse, adult human, and the like. Pancreas organoid tissue cultures may employ a stem cell culture condition (niche factors) including media components such as one or more of EGF, Noggin, R-spondin, Wnt-3A, FGF10, nicotinamide, and the like, depending on source. The differentiation culture condition may include EGF, Noggin, R-spondin, Wnt-3A, and the like, depending on source.

As a further example, liver organoid tissue cultures may be derived from a source such as, e.g., adult mouse, adult human, and the like. Liver organoid tissue cultures may employ a stem cell culture condition (niche factors) including media components such as one or more of EGF, Noggin, R-spondin, Wnt-3A, FGF10, HGF, nicotinamide, and the like, depending on source. The differentiation culture condition may include EGF, Noggin, R-spondin, Wnt-3A, FGF10, TGF-beta inhibitor, Notch inhibition, BMP7, and the like, depending on source.

The term “tumoroid” refers to three dimensional (3D) multicellular in vitro tissue culture aggregates composed of one or more cell types typically derived from primary tumors harvested from oncological patients and can mimic human tumor microenvironment. Tumoroids may be useful for studies on novel cancer drugs or for use in precision medicine in the field of oncology. Cancer cell lines may be, for example, bladder, breast, colon, hematopoietic and lymphoid, liver, lung, ovary, prostate, skin, and the like.

The term “stem cells” refers to undifferentiated cells that have the potential to develop into many different cell types that carry out different functions. Pluripotent stem cells, such as those found in embryos, can give rise to any type of cell such as those in brain, bone, heart, and skin. Some human adult cells can be reprogrammed into embryonic stem cell-like state called induced pluripotent stem cells (iPSCs). Multipotent stem cells, for example, found in adults or in babies umbilical cords, may develop into the cells that make up the organ system that they originated from. When grown under certain cell culture conditions, pluripotent stem cells can remain undifferentiated. To generate differentiated cells, the chemical composition of the culture medium may be changed, the surface of the culture dish may be altered, or the cells may be modified by forcing expression of certain genes.

The term “production cell line” refers to a cell line that can be used for production of enzymes, vaccines, monoclonal antibodies, cytokines, peptides, therapeutic toxins, clotting factors, Fc-fusion proteins, or hormones, and the like. The production cell line can be a prokaryotic or eukaryotic production cell line. The production cell line may be any production cell line appropriate for protein or virus production. Prokaryotic production cell lines may include any appropriate prokaryotic cell line. The prokaryotic production cell lines may include any appropriate bacterial cell line (e.g., Escherichia coli). Eukaryotic production cell lines may include Chine Hamster Ovary (CHO) cell lines, murine myeloma cell lines (e.g., NS0, Sp2/0), murine C127, human embryonic kidney (HEK) cell line such as a HEK293 cell line, an human fibrosarcoma cell line such as a HT-1080 cell line, human embryonic retinal cell lines such as a PER.C6 cell line, a baby hamster kidneys (BHK) cell line such as a BHK21 cell line, yeast cell lines (e.g., Saccharomyces cerevisiae, Pichia pastoris), insect cell lines infected with viral vector baculovirus (baculovirus-insect cell expression system), e.g., a Sf9 cell line, plant cells.

The term “secreted factor” or “secreted factors” refers to factors that may be secreted from the target cell during the automated cell culture applications of the present disclosure. The secreted factors may be any secreted factor including hormones, metabolites, enzymes, vaccines, monoclonal antibodies, cytokines, peptides, therapeutic toxins, clotting factors, or Fc-fusion proteins.

A “trans well” culture, also known as an in-direct co-culture, may include vertically arranged wells having a lower compartment and an upper compartment such as a trans well insert, separated by, for example, a 0.4 uM microporous membrane to minimize direct contact between producer cells and target cells. In some embodiments, the tissue culture methods of the disclosure do not employ trans well culture.

Different media components may be required for each type of source cells used, and the type of differentiation to be achieved. Growth factors such as EGF, Noggin (NOG), R-spondin (RSPO1), HGF, BMP, FGF, and the like may be essential components of organoid media. The tissue culture media may comprise growth factors. The growth factors may be generated by the feeder cells. The growth factors may be recombinant growth factors. The recombinant growth factor proteins for organoid culture may include, for example, recombinant human EGF protein, recombinant HGF proteins such as, for example, human HGF protein, cynomolgus HGF protein, human FGF10, human Noggin/NOG protein, human RSPO1 protein, human BMP-2 protein, and the like. Additional recombinant growth factors for organoid culture may include, for example, EGF, FGF2, FGF7, FGF9, FGF10, HGF, NOG, RSPO1, RSPO3, Activin A, BMP2, and BMP4, and the like. The tissue culture media, or recombinant growth factor proteins for organoid culture, may be commercially available from, for example, Sino Biological, Inc., or Thermo Fisher Scientific.

3D cellular models like organoids and spheroids may be cultivated in a tissue culture media comprising a hydrogel, such as in a hydrogel dome within the media.

The term “hydrogel” or “hydrogels” refers to an extracellular matrix useful for culturing organoids. The hydrogel may include murine EHS sarcoma matrix, for example, available commercially as Matrigel (Corning), Cultex (Trevigen), Geltrex (Gibco), collagen type I, fibrin, hyaluronic acid (HA), gelatin methacrylate (GelMA), decellularized matrices, or biopolymers such as alginate, silk, nanocellulose; engineered materials such as polyethylene glycol (PEG), self assembling peptides such as RADA16/PuraMatrix bQ13, poly(lactic/(co)glycolic) acid, polycaprolactone, polyacrylamide, oligo (ethylene glycol)-substituted polyisocyanopeptides, ELP (elastin-like protein), or combinations of these polymers.

### Horizontal Co-Culture for Cell/Organoid Feeding

A method for horizontal co-culture for cell/organoid feeding is provided. The well unit 100 as shown in FIG. 1A may be employed. The method comprises allowing one or more target cells to adhere to the bottom of a primary well 112 of a well unit 100 of a separation well microplate comprising incubating without mixing, the primary well separated from a secondary well 115 of the well unit of the separation well microplate by a closed at least one microchannel; allowing one or more feeder cells to adhere to the bottom of the secondary well comprising incubating without mixing; opening the closed at least one microchannel; rocking the separation well microplate to allow mixing and media exchange between the primary well 112 and the secondary well 115 by gravity flow through the open at least one microchannel 118 as shown in FIG. 1B; detaching target cells from the primary well 112; and settling the detached target cells in the primary well 112 away from the open microchannel. The method may further include mixing the detached target cells and allowing to further settle in the primary well away from the open microchannel; washing the settled target cells; and collecting the washed target cells via a liquid handler. The washing may include suspending of settled target cells comprising introducing and removing of a liquid medium, and the washing can optionally be repeated multiple times. In some embodiments, the allowing of the one or more target cells to adhere to the bottom of the primary well 112 and the allowing of the one or more feeder cells to adhere to the bottom of the secondary well 115 are performed simultaneously or essentially simultaneously during an initial incubation period.

Initially the at least one microchannel may be closed, for example, by an airgap within or adjacent to the microchannel. The closed at least one microchannel 118 may be opened by eliminating the airgap after at least a part of the initial incubation period. For example, the at least one microchannel may be opened by using a pipette as a pump to force liquid media exchange between the primary well and the secondary well. To use the pipette as a pump, the outer surface of the pipette is engaged with a perimeter of secondary well as to form a seal. Liquid in the pipette is then forced into and through the microchannel 118, dislodging the airgap.

The detaching of the target cells from the primary well 112 may comprise exposing the target cell to a dissociation reagent to detach the target cells without detaching the feeder cells, optionally wherein the dissociation reagent comprises EDTA. The dissociation reagent may comprise a detachment enzyme in a physiological buffer. For example, the detachment enzyme may be selected from the group consisting of a trypsin, a collagenase, an elastase, a catalase, a superoxide dismutase, and a dispase, or the like.

In some examples, the target cells may be stem cells or organoids. In some examples, the feeder cells may be fibroblasts. In some examples, the target cells are embedded in a hydrogel disposed in the primary well of the well unit.

Detection of Secreted Factors from Cells Allowing Kinetic Measurement within a Single Microplate

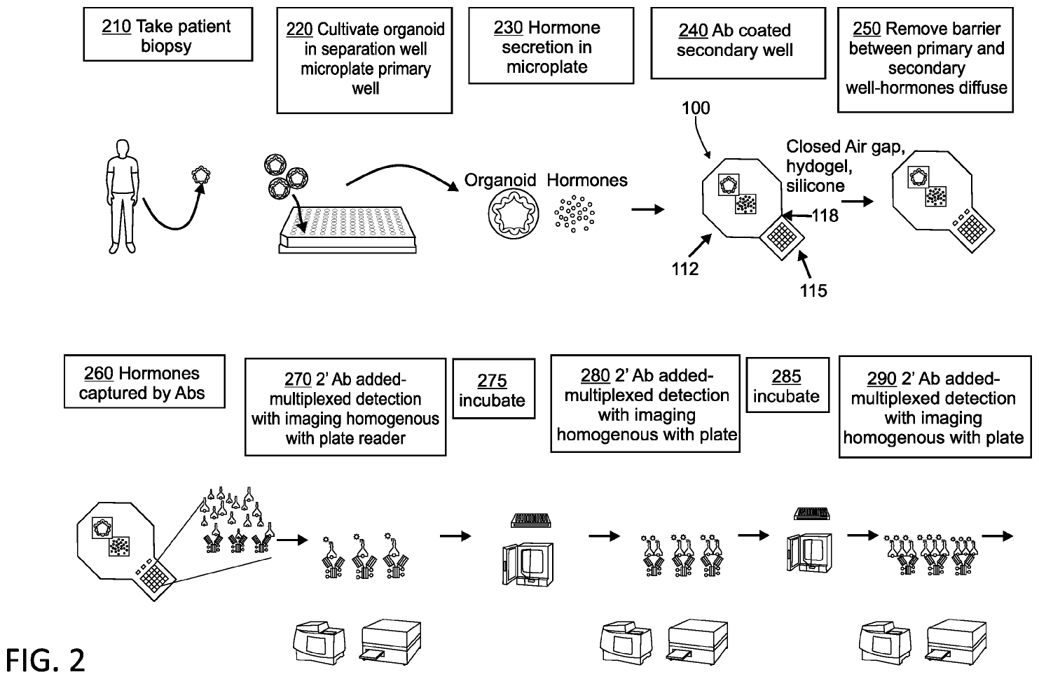

Target cells may produce secreted factors. For example, as shown in FIG. 2, gut organoids may secrete hormones 230, or a production cell line such as CHO cells may secrete antibodies. A method is provided for detection of multiple secreted factors from cells allowing kinetic measurement within a single microplate over time. For example, multiple secreted factors may be detected at different time points (kinetic measurement) 270, 280, 290 after formation of organoids from stem cells or organoid fragments.

FIG. 2 illustrates a method for detecting secreted factors from target cells comprising kinetic measurement over time within a single separation well microplate. In some embodiments, the secreted factors may be selected from hormones, antibodies, cytokines, chemokines, or growth factors. A patient biopsy sample 210 may be obtained which may be source for target cells, for example, multicellular target cells such as organoids or tumoroids; other sources of organoids may be utilized such as organoid fragments or stem cells. Cultivation of target cells such as organoids can be performed in a primary well of a well unit of a separation well microplate 220. The organoid may secrete hormones 230 into the liquid media. During an initial incubation period the primary well is separated from the secondary well by at least one closed microchannel, and the secondary well bottom is coated/spotted with primary antibodies (Ab) specific for the secreted factors 240. After an initial incubation period, the barrier in the microchannel 250 is removed to allow diffusion of the secreted factors between the primary well and the secondary well through the open microchannel. The secreted factors are captured 260 by the primary antibodies in the secondary well. Labeled secondary antibodies (2′ Ab) specific for the primary antibodies or the captured secreted factors are added to the secondary well at a first time point, and bound 2′Abs are detected in the secondary well with a microplate reader 270. The diffusion of the secreted factors may comprise rocking the separation well microplate to allow diffusion of the secreted factors between the primary well and the secondary well by gravity flow through the open microchannel. The detecting may comprise multiplexed detection with imaging, homogenous with the plate reader.

As shown in FIG. 2, after further incubating the target cells in the microplate 275, further labeled secondary antibodies (2′Ab) specific for the primary antibodies or the captured secreted factors are added at a second time point and detected with a microplate reader 280. Optionally, after further incubating the target cells in the microplate 285, further labeled secondary antibodies (2′Ab) specific for the primary antibodies or the captured secreted factors are added at a third time point and detected with a microplate reader 290. Further sequential incubation and detection operations may be performed over fourth, fifth, sixth, seventh, and further time points. This method allows for kinetic measurement of secreted factors over time in the same separation well microplate used for cultivation of the target cells.

The labeled antibodies may be labeled with any appropriate label such as a fluorochrome. Different antibodies can be differentially labeled with different fluorophores. The primary antibodies (Ab) and the labeled secondary (2′ Ab) antibodies may be purchased commercially such as from, for example, Beckman Coulter, Inc., Abcam, or Novus Biologicals. The labeled secondary antibodies (2′ Ab) may be prepared using antibody labeling methods known in the art, for example, such as via N-hydroxy succinimide esters (NHS esters), heterobifunctional reagents, or carbodiimides.

In some embodiments, the target cells are organoids that may be derived from a target tissue selected from the group consisting of biopsy sample from a patient, stem cells, or organoid fragments. The organoids may be cultivated within a hydrogel dome in the primary well of the well unit. For example, the organoids may be grown from stem cells or from organoid fragments in the primary (culture) well within a hydrogel dome (such as Matrigel).

Initially the primary and secondary wells are separated by, e.g., air captured within the microchannels (or by other means)—avoiding any nonspecific binding on the detection array. The at least one closed microchannel may be closed by a barrier comprising an airgap, hydrogel seal, or silicone seal, within or adjacent to the microchannel, optionally during at least a part of the initial incubation period. When the microchannel is closed by a barrier comprising an airgap, the opening of the closed microchannel may comprise eliminating the airgap after at least a part of the initial incubation period; optionally comprising using a pipette as a pump to force liquid media exchange between the primary well and the secondary well. For example, the airgap may be eliminated after the organoids have been fully developed by using the pipette as a pump (sealing the pipette tip to the feeding well) to force liquid flow between the primary and secondary wells. For example, the airgap may be eliminated by aspirating liquid through the microchannel between the primary and secondary wells. In another example, gravity flow may be used to exchange media between the wells to transport the excretion products of the organoids to the secondary well comprising the spotted/coated Abs.

The secondary well may be spotted/coated with a plurality of different primary antibodies specific for a plurality of different secreted factors, and the primary antibodies may be arranged in the bottom of the secondary well in a detection array. The detected secreted factor type may be positioned on specific areas of the detection array. The quantity of hormones can be measured by the strength of fluorescence signal after washing. In some embodiments, the different time points can be recorded by the help of different fluorophores—for example, a different fluorescence color for every time point. The concentration of capture antibodies (Ab) can be normalized, for example, by using a different fluorophore.

The measurement can be done with a microscope or with a plate reader. A plate reader has the advantage of allowing a high dynamic range but may require about 1 mm² for every detection array point. An imager can detect a higher number of array points being able to observe much smaller array points but may have a more limited dynamic range.

Functionalized Surface with Antibodies for Stem Cell Isolation

Some organoid types require passaging in a way to break up the organoids to the single cell level. In this case it is important to capture the desired (adult) stem cells to be able to recreate a new organoid.

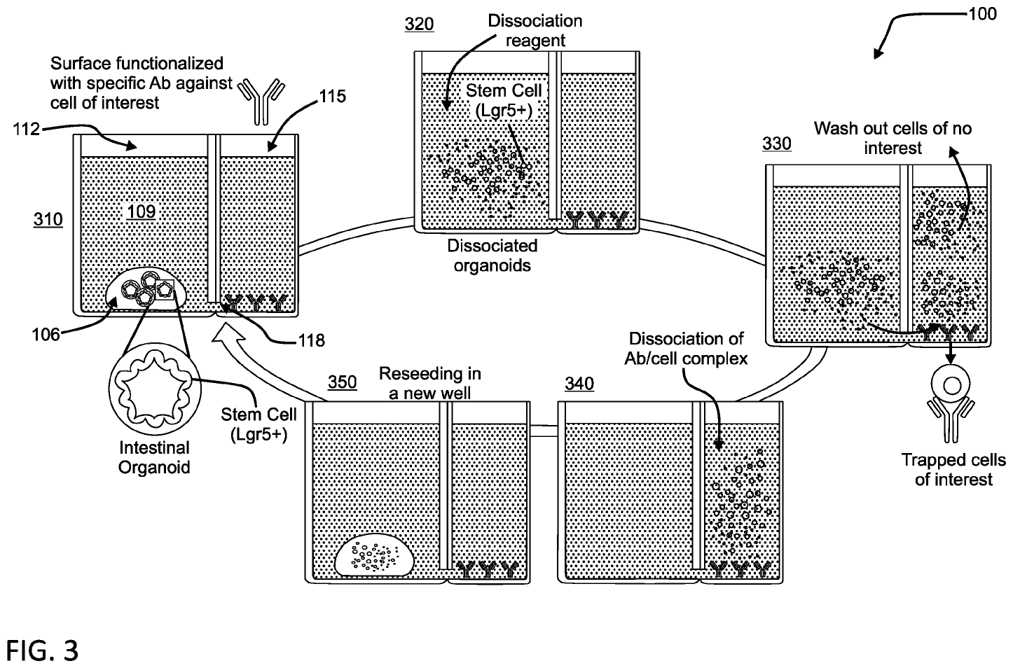

Another embodiment of the disclosure provides a method for organoid passaging using single cells passaging and shall have a secondary well having a surface functionalized with antibodies to specifically capture stem cells during the washing process at the end of every cultivation cycle. FIG. 3 shows an exemplary method for passaging in a way to break up the organoids to the single cell level with isolation of desired stem cells. Operation 310 comprises cultivating organoids in a well unit of a separation well microplate having a secondary well 115 coated or surface functionalized with immobilized primary antibodies (Ab) specific for a stem cell (e.g., Lgr5+), a primary well 112 containing the organoids 103 (e.g., intestinal organoids) within a hydrogel dome 106 disposed at the bottom surface of the primary well 112, and initially a closed microchannel 118. A liquid medium 109 is also illustrated in both the primary well section 112 and the secondary well section 115 of the well unit 100. The liquid medium 109 can comprise appropriate growth factors and supplements to support growth of the organoids 103. Operation 320 comprises breaking down the dome of hydrogel 106 into a plurality of hydrogel fragments 320 or otherwise liquid form to release the organoids 103 into the liquid media 109.

The present disclosure provides an automated process 320 for breaking the hydrogel dome. In the prior art breaking the hydrogel dome is typically performed manually using a pipet to repetitively inject/spear the dome randomly to help break it up, for example, about 20 times.

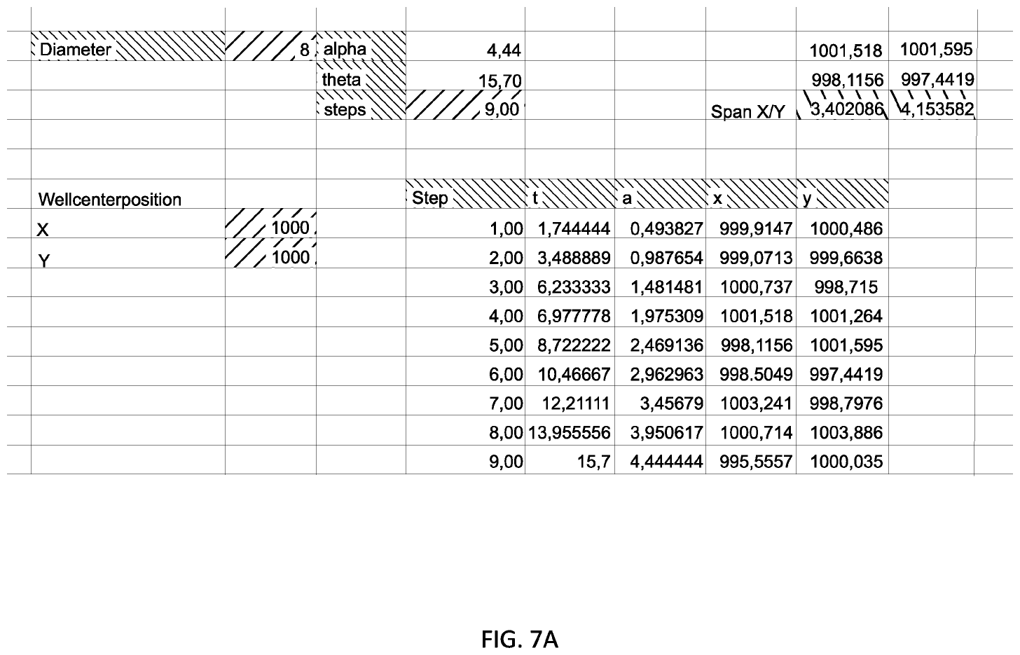

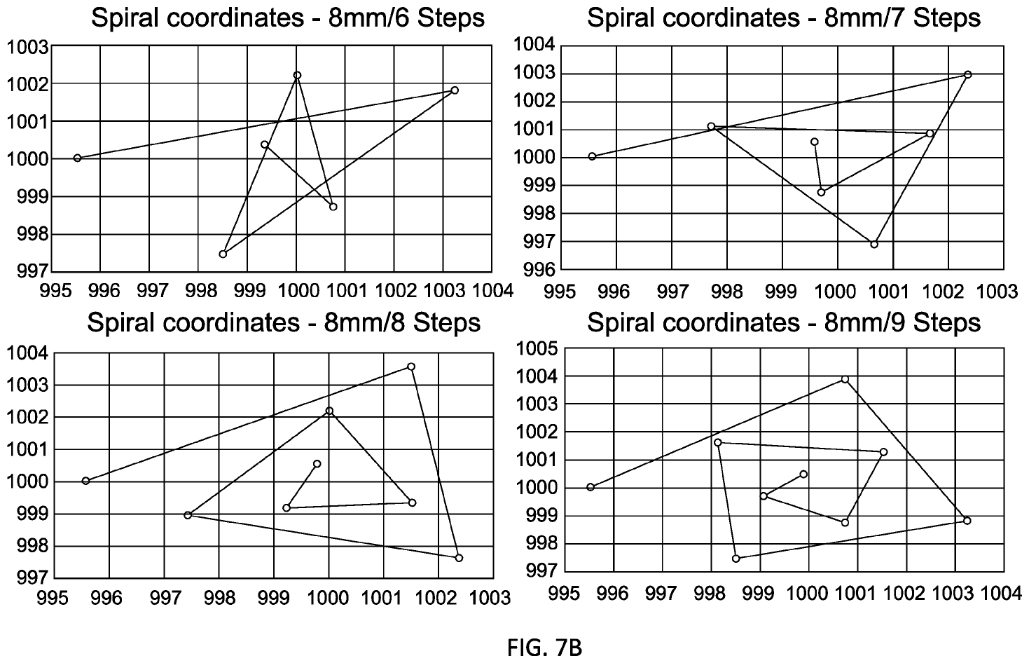

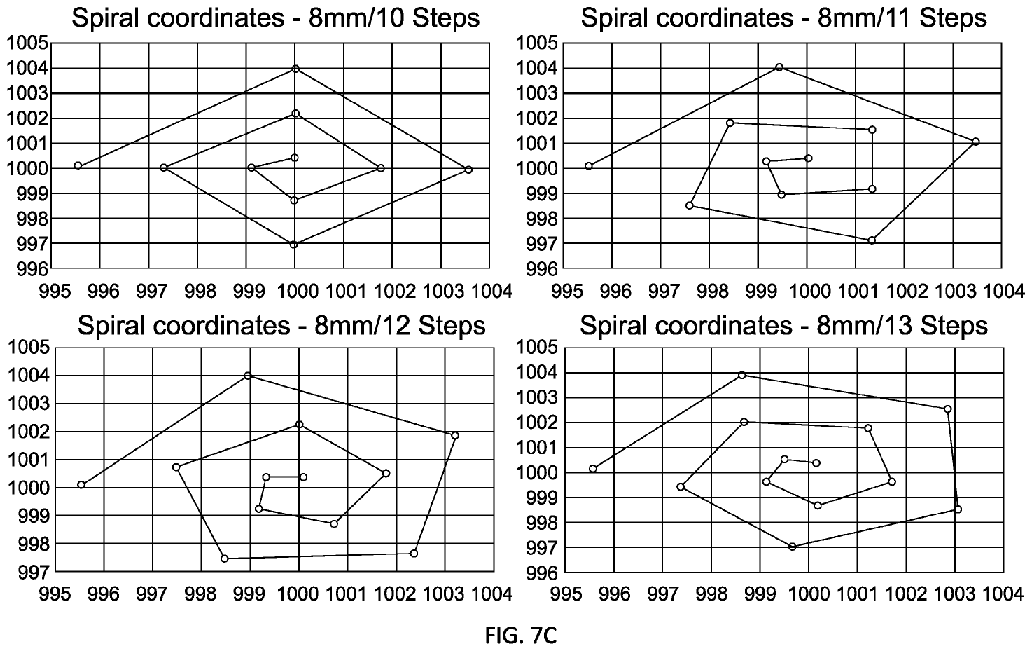

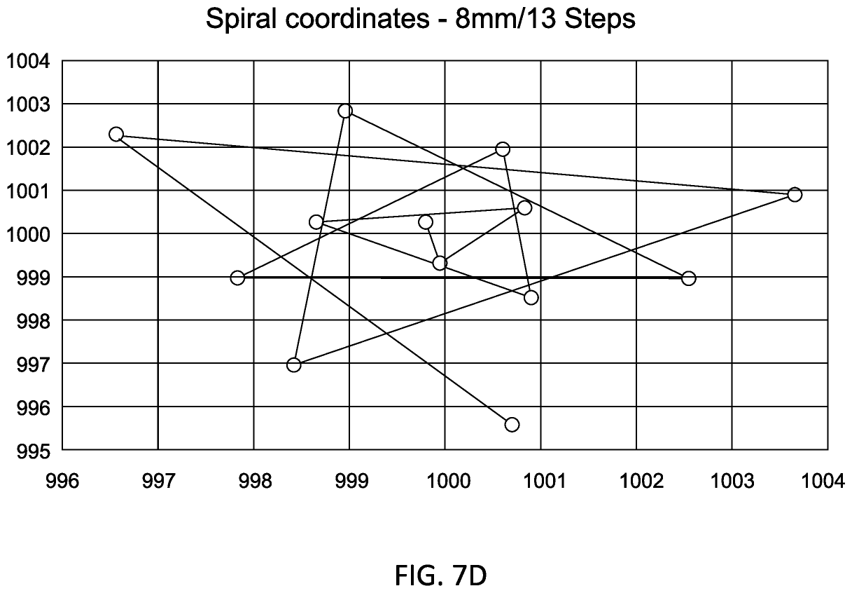

An automated method 320 of breaking a hydrogel dome is provided, the method comprising cultivating a first cell in a hydrogel dome in a liquid media in a well of a microplate over an initial incubation period of time to provide multicellular target cells; puncturing the hydrogel dome at or near the x,y center position of the dome in the well with a pipette tip associated with a liquid handler after the initial incubation period of time; moving the pipette tip through the dome in a multiplicity of steps, each step comprising an X,Y predetermined position within the well to break the hydrogel dome and optionally liquify the hydrogel dome to free the target cell from the hydrogel dome within the well. The target cells may then be isolated. The automated pipette tip may be positioned at or near the center of the hydrogel dome after culturing a target cell. In some cases, the first cell may be a target cell or a stem cell. The liquid handler may aspirate the pipette at one or more, two or more, or each of the multiplicity of steps. For example, the pipette tip may be positioned at the center of the hydrogel dome, or within 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, or within 0-2 mm, or 0.01-1.5 mm of the center of the x,y center of the hydrogel dome. Each of the X, Y predetermined positions within the well may be calculated comprising:
X(stepnumber)=Xwellcenter+alpha/steps*stepnumber*COS(theta/steps*stepnumber); and
Y(stepnumber)=Ywellcenter+alpha/steps*stepnumber*SIN(theta/steps*stepnumber), wherein alpha=dome diameter (mm)/1.8; steps=number of steps for moving the pipette tip through the hydrogel dome; Xwellcenter=x position of dome center within the well; Ywellcenter=y position of dome center within the well; and theta=a constant value between 5 and 50. In some cases, the number of steps is an integer from 2-20, 3-18, 4-17, 5-15, 6-12, or 8-10. In some cases, theta is a constant from 5-50, 10-45, 15-40, 15-35, or any number in between. In some cases theta, is selected from the group consisting of 5, 10, 12, 13, 14, 15, 15.7, 16, 17, 18, 20, 22, 24, 26, 28, 30, 32, 34, 36, 38, 40, 45, or 50, or any number in between. The x,y center position of the hydrogel dome in the well may be determined based on the original seeding position in the well, volume of hydrogel deposited in the well, and/or imaging of the hydrogel dome. The pipette tip may be used to puncture the dome, and/or aspirate within the dome. For example, the pipette may be used to apply, remove, or aspirate a liquid or air within the dome. Inputs to the system may include seeding position x,y coordinates in the well and the known volume of the dome. Another input may be the number of steps of dome pipette punctures or pipette aspirations. The diameter of the dome may be calculated based on the volume. The diameter of the dome may be determined by imaging. The diameter of the dome may be any appropriate diameter. For example, the diameter of the dome may be 1-12 mm, 2-10 mm, 3-8 mm, or 3-6 mm. The number of steps may be any appropriate number. For example, the number of steps may be 1, 2, 3, 4, 5, 6, 7, 8, 9, 10, 11, 12, 13, 14, 15, 16, 17, 18, 19, or 20, or 2-20, 3-18, 4-17, 5-15, 6-12, or 8-10. As illustrated in FIG. 7A, inputs include the diameter of the hydrogel dome in this case 8 mm, the number of steps is 8, and well center position X, Y coordinates. In some cases, the number of steps/positions may be minimized to effectively break the hydrogel in order to minimize process time. The output may include an automated sequence/pattern of puncturing/aspirating positions within the dome with a pipette by the liquid handler. In some embodiments, the pattern of the puncturing/aspirating positions may be a spiral pattern. In some embodiments, the pattern of the puncturing/aspirating positions is a star pattern. In some embodiments, the pattern of the puncturing/aspirating positions is a zig-zag pattern. At each position, the automated pipette may aspirate within the dome. The pipette tip may be dragged beneath the surface of the dome between steps/positions. For example, the pipette tip position in the Z axis may be maintained below the surface of the hydrogel dome between steps/positions for efficient breaking of the dome. Between steps, the pipette tip position in the Z axis may be maintained at or near the bottom of the hydrogel dome. For example, the pipette tip may be maintained below the surface, within 0.5, 1.0, 1.5, or 2 mm of the surface, at or within 0.5, 1.0, 1.5, or 2 mm near the center of the Z axis, at the bottom, or within 0.5, 1.0, 1.5, or 2 mm of the bottom of the dome between steps or during aspirating. The Z axis position may be determined by the volume of the hydrogel dome or by imaging the well. Exemplary FIGS. 7B-7D shows an output series of spiral X, Y coordinates of the automated pipettor within the well using inputs of well center position, 8 mm dome diameter, and 6-13 steps, using theta of 15.7 (FIGS. 7B-C) or 30 (FIG. 7D) resulting in effective breaking of the hydrogel dome. For example, the solid hydrogel 106 can be transformed into a liquid, thereby separating the organoid 103 from the hydrogel 106. The hydrogel may be transformed into a liquid by, e.g., harsh pipetting, or shear forces of a liquid handler, with or without a decrease in temperature, for example, to about 4 deg C. to about 10 deg C, or about 10 deg C, from incubation temperature (about 37 deg C). A dissociation reagent can be added to the primary well 112 in order to dissociate the organoid into the stem cells and the plurality of additional cell types. Following formation of the dissociated organoids, the microchannel 118 is opened, for example, by removing the airgap. Liquid aspiration via the secondary well is performed after the organoids have been broken up. Stem cells are allowed to be captured by the primary antibodies (Ab) within the secondary well 115. Operation 330 comprises washing the well unit 100 to remove undesirable cells and debris. Operation 340 comprises dissociating the isolated stem cells from the antibody/cell complex in the secondary well. Operation 350 comprises reseeding the isolated stem cells into a new hydrogel dome 106 within a new primary well 112.

Alternatively, at operation 310 the secondary well 115 may be coated or surface functionalized with an immobilized capture reagent such as a biotin capture reagent or an avidin capture reagent. With respect to a biotin immobilized capture reagent, after treating organoids with dissociation reagent at 320, the dissociated organoids may be treated in solution with an antibody specific for the stem cell of interest conjugated to a reagent having strong affinity for the capture reagent, such as a streptavidin (e.g., antibody-streptavidin conjugate) to form a stem cell-antibody-streptavidin complex. The microchannel 118 is opened by removing the removable barrier, and stem cell-antibody-streptavidin complex is allowed to bind to the immobilized biotin in the secondary well 115. After washing away unbound cells and debris in operation 330, the stem cells may be dissociated from the complex. Remaining operations 340 and 350 may be performed as provided herein. Alternatively, prior to removing the removable barrier an antibody conjugate comprising a stem cell-specific primary antibody conjugated to a streptavidin may be allowed to bind to the immobilized biotin capture reagent.

Functionalized Scaffold with Antibodies for Stem Cell Isolation

Some organoid types require passaging in a way to break up the organoids to the single cell level. Additional methods are provided to capture the desired (adult) stem cells to be able to recreate a new organoid.

Another embodiment of the disclosure provides a method for organoid passaging using single cells passaging and shall have a secondary well including a scaffold that is coated or surface functionalized with immobilized antibodies to capture specific stem cells during the washing process at the end of every cultivation cycle.

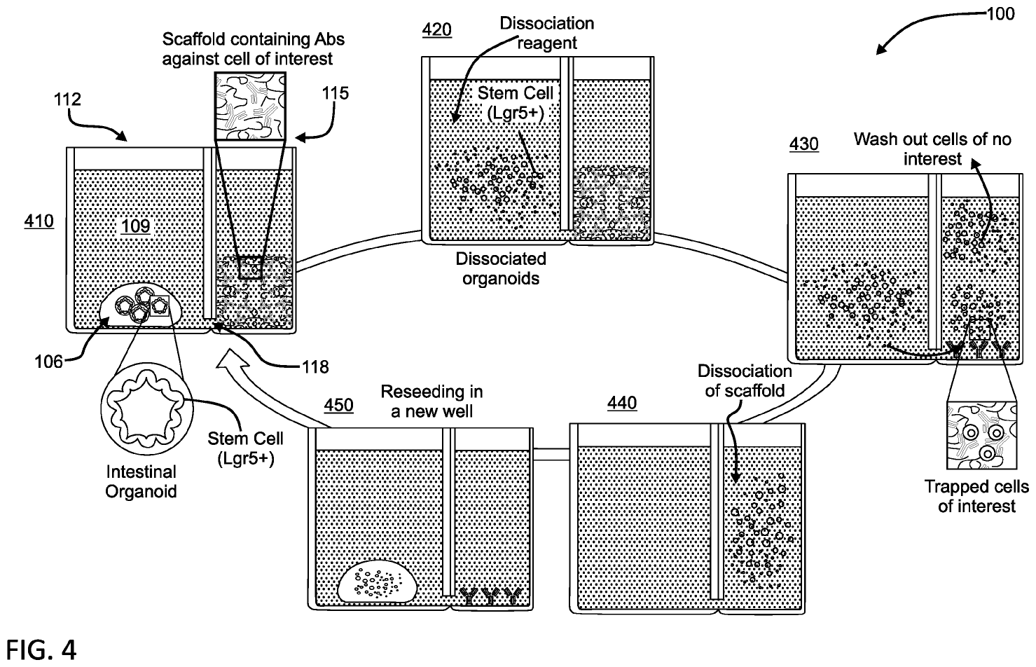

FIG. 4 shows an exemplary method for passaging in a way to break up the organoids to the single cell level with isolation of desired stem cells. Operation 410 comprises cultivating organoids in a well unit of a separation well microplate comprising a primary well 112 containing the organoids 103 (e.g., intestinal organoids) within a hydrogel dome 106 disposed at the bottom surface of the primary well 112, with a closed microchannel 118 between primary and secondary wells, and a secondary well 115 having a porous scaffold coated or surface functionalized with primary antibodies (Ab) specific for a stem cell (e.g., Lgr5+). A liquid medium 109 is also illustrated in both the primary well section 112 and the secondary well section 115 of the well unit 100. The liquid medium 109 can comprise appropriate growth factors and supplements to support growth of the organoids 103.

Operation 420 comprises breaking down the dome of hydrogel 106 into a plurality of hydrogel fragments 320 or otherwise liquid form to release the organoids 103 into the liquid media 109. For example, the solid hydrogel 106 can be transformed into a liquid, thereby separating the organoid 103 from the hydrogel 106. The hydrogel may be transformed into a liquid by, e.g., harsh pipetting, or shear forces of a liquid handler, with or without a decrease in temperature, for example, to about 4 deg C. to about 10 deg C, or about 10 deg C, from incubation temperature (about 37 deg C). A dissociation reagent is added to the primary well 112 in order to dissociate the organoid into the stem cells and the plurality of additional cell types. Following formation of the dissociated organoids, the microchannel 118 is opened, for example, by removing the airgap. Liquid aspiration via the secondary well is performed after the organoids have been broken up. Stem cells are allowed to be captured by the primary antibodies (Ab) within the secondary well 115.

Operation 430 comprises washing the well unit 100 to remove undesirable cells and debris. Operation 440 comprises dissociating the isolated stem cells from the antibody/cell complex in the secondary well. Operation 450 comprises reseeding the isolated stem cells into a new hydrogel dome 106 within a new primary well 112.

### Magnetic Beads for Stem Cell Isolation

Some organoid types require passaging in a way to break up the organoids to the single cell level. Magnetic beads coated with antibodies specific for stem cells may be added to the primary well after breaking up the organoids to capture the desired (adult) stem cells to be able to recreate a new organoid.

A magnet is placed under the primary well during the washing operations to retain the (adult) stem cells within the primary well. The antibody binding can be broken by several means, such as for example, enzymatic means, such as by trypsin, or by adding a linker that can be digested by an enzyme.

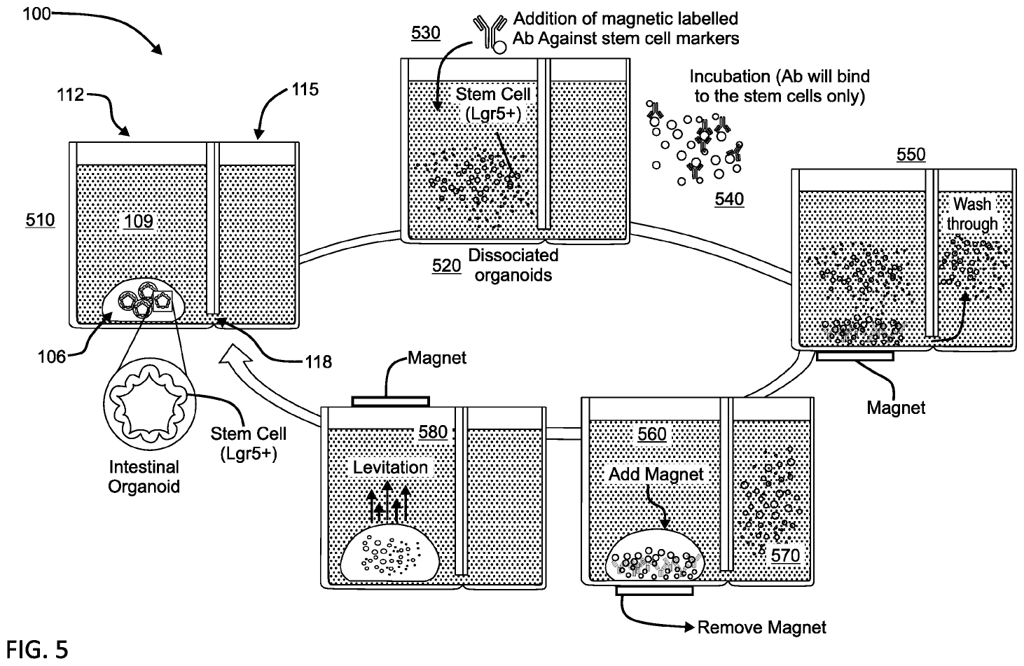

FIG. 5 shows an exemplary method for passaging in a way to break up the organoids to the single cell level with isolation of desired stem cells. Operation 510 comprises cultivating organoids in a well unit of a separation well microplate comprising a primary well 112 containing the organoids 103 (e.g., intestinal organoids) within a hydrogel dome 106 disposed at the bottom surface of the primary well 112, with a closed microchannel 118 between primary and secondary wells. A liquid medium 109 is also illustrated in both the primary well section 112 and the secondary well section 115 of the well unit 100. The liquid medium 109 can comprise appropriate growth factors and supplements to support growth of the organoids 103.

Operation 520 comprises breaking down the dome of hydrogel 106 into a plurality of hydrogel fragments 320 or otherwise liquid form to release the organoids 103 into the liquid media 109. For example, the hydrogel 106 can be transformed into a liquid, thereby separating the organoid 103 from the hydrogel 106. The hydrogel may be transformed into a liquid by, e.g., by shear forces of a liquid handler or harsh pipetting, with or without a decrease in temperature, for example, to about 4 deg C. to about 10 deg C, or about 10 deg C, from incubation temperature (about 37 deg C). A dissociation reagent is added to the primary well 112 in order to dissociate the organoid into the stem cells and the plurality of additional cell types.

Operation 530 comprises adding magnetic bead labeled antibodies specific for stem cell markers. Operation 540 comprises allowing the magnetic beads coated with the antibodies to incubate with the dissociated organoids to allow binding of the desired stem cells. Operation 550 comprises applying a magnet to the bottom of the primary well, opening the microchannel 118, and washing the unbound cells and debris through the secondary well by, for example, aspirating the liquid media with a liquid handler. Optionally operation 550 may further include transferring the washed captured cells to a new well unit of a new separation well microplate comprising removing the magnet to allow transfer of the captured cells to the new well unit of the new separation well microplate. The magnet shall be reapplied under the new well unit before continuing with operation 560. Operation 560 comprises adding new hydrogel to the primary well such that the stem cells and magnetic beads are embedded in the hydrogel, and removing the magnet from under the primary well. Operation 580 comprises placing a magnet at the top of the primary well to levitate the stem cells in the hydrogel. Immuno-therapy of tumoroids and organoids for toxicology studies

CAR T-Cell and other cells therapies can be beneficial for cancer treatment. Migration, invasion of the tissues and tumor specific cell killing may be to be evaluated. This assay comprises several readouts that can be beneficial for the evaluation of the immune-therapies.

Another embodiment of the disclosure is a method for organoid/tumoroid culturing and to observe how fibroblasts, macrophages or other cells migrate though the microchannel to the main organoid dome. The effects of these cells can be evaluated by observing phenotypically changes in the organoids/tumoroids.

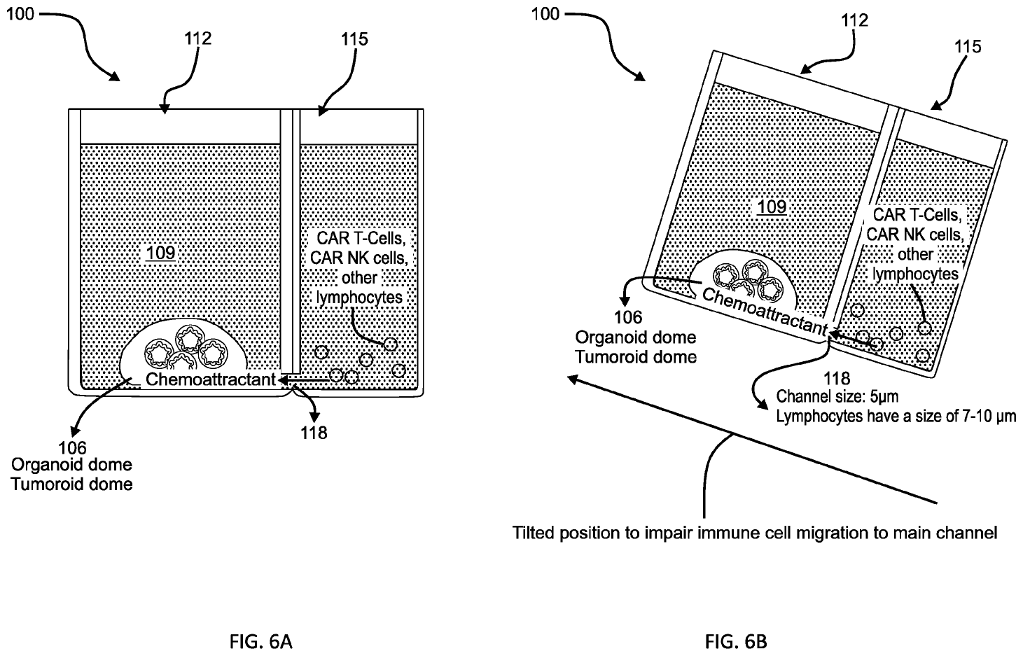

FIGS. 6A and 6B illustrate methods for evaluating cell (e.g., lymphocyte) functionality and toxicology studies in a single separation well microplate. FIG. 6A shows a view of a well unit 100 of a separation well microplate comprising multicellular target cells (e.g., organoids) in a hydrogel dome 106 in liquid media 109 in the primary well 112 of a microwell unit of a separation well microplate. The secondary well 115 comprises chimeric antigen receptor T-cells (CAR-T cells), CAR natural killer (CAR-NK cells), or other lymphocytes. The microchannel 118 may have a channel size of 5 microns while the lymphocytes have a size of 7-10 microns. Organoids may emit a chemoattractant which may cause immune cell migration into the primary chamber, depending on microchannel size. FIG. 6B shows effect of tilting the separation well microplate, wherein a tilted position may be employed to impair immune cell migration to the primary well 112.

### Additional Assays

In addition to the assays described above, the structure of the separation well microplate allows for at least two major advantages for cell assay development and increasing complexity of cell assays.

A first major advantage is the ability to allow cell movement or migration between the primary and secondary wells of the well unit. The distance for movement or extension is short, while primary and secondary wells can be efficiently separated. The separation well microplate does not have to include matrix or forced liquid flow.

Many application methods illustrate this advantage. For studying angiogenesis the tumor micro-tissue or growth factors will be present in one well of the well unit, while the layer of endothelial cells will be in other well of the well unit. Endothelial cells would have the ability to migrate to the first chamber, and may form angiogenesis sprouts.

For studying neurogenesis-neuro-regeneration the effects of growth factor or neurotoxic agents on neurite outgrowth may be developed. Neurons will be placed into one chamber, and will extend outgrowth into second chamber upon growth factors present in that chamber. The process will depend on treatment agents. The advantage is the ability to study outgrowth in given direction.

For studying tumor cell evasion/invasion, migration, wound healing, normal or tumor cells could be studied for their motility into different chamber. Migrated cells can be more easily and accurately counted in different cells of the cell unit in comparison with traditional cell migration assay.

For studying T cell invasion, or monocyte migration, immune cells could be placed into the chamber, while chemotaxis factors of micro-tissues will be placed into the second chamber. Immune cells will move to the other chamber upon chemotaxis.

A second major advantage of structure of the separation well microplate is the ability to perform co-culture and study cross-talk of different cell types and micro-tissues. In this model cells representing different tissues would be separated in different wells of the well unit, while the microplate will allow media exchange between the primary and secondary wells of the well unit, so the effects of added compounds and secreted factors may be studied. For example: liver cells may secrete factors that would affect cardiac cells. Endothelial cells may secrete cytokines in response to inflammation that would attract immune cells, or may cause damage of liver cells or neuronal cells (liver cirrhosis, stroke models). Some other applications may include: multi-tissue toxicity evaluation of organ tissue pairs such as liver-brain, liver-heart, kidney-liver, intestinal-liver, etc. Differentiation and self-renewal of stem cells in response to compounds, toxins, growth factors, inflammation, metabolic switching may be studied. In some embodiments, the evaluation of secreted factors only or movement of cells could be applied by using either a horizontal slit configuration to allow cells to migrate, or a vertical wider slit configuration for feeding and media-growth factor exchange.

1. A method for co-culturing target cells, the method comprising
introducing one or more target cells to a primary well of a well unit of a separation well microplate and initially incubating in a liquid media without mixing to allow the target cells to adhere to the bottom of the primary well, the primary well separated from a secondary well of the well unit of the separation well microplate by at least one closed microchannel comprising a removable barrier;
introducing one or more feeder cells to the secondary well and initially incubating without mixing in a liquid media to allow the feeder cells to adhere to the bottom of the secondary well;
removing the removable barrier to open the at least one closed microchannel;
rocking the separation well microplate to allow mixing and media exchange between the primary well and the secondary well by gravity flow through the open microchannel;
detaching target cells from the primary well; and
settling the detached target cells in the primary well away from the open microchannel.

2. The method of claim 1, further comprising
mixing the detached target cells and allowing the detached target cells to further settle in the primary well away from the open microchannel;
washing the settled target cells; and
collecting the washed target cells via a liquid handler.

3. The method of claim 1, wherein the introducing one or more target cells to the primary well and the introducing one or more feeder cells to the secondary well are performed simultaneously or essentially simultaneously.

4. The method of claim 3, wherein the at least one closed microchannel is closed by a removable barrier comprising an airgap, hydrogel seal, or silicone seal, within or adjacent to the microchannel.

5. The method of claim 4, wherein the removable barrier comprises an airgap and the removing the removable barrier comprises eliminating the airgap after at least a part of the initial incubation period.

6. The method of claim 1, wherein the detaching comprises exposing the target cell to a dissociation reagent to detach the target cells without detaching the feeder cells.

7. The method of claim 6, wherein the dissociation reagent comprises a detachment enzyme in a physiological buffer.

8. The method of claim 1, wherein the target cells are stem cells or organoids.

9. The method of claim 1, wherein the feeder cells are fibroblasts.

10. The method of claim 1, wherein the target cells are embedded in a hydrogel dome disposed in the primary well of the well unit in the liquid media.

11. The method of claim 1, wherein the at least one closed microchannel is located between the bottom surface of the well unit and a bottom portion of a shared sidewall of the primary well and the secondary well.

12. The method of claim 1, wherein the height of the at least one microchannel is in a range of about 10 microns to about 100 microns or about 10 microns to about 75 microns.

13-55. (canceled)

56. The method of claim 3, wherein the at least one closed microchannel is closed by a removable barrier comprising an airgap, hydrogel seal, or silicone seal, within or adjacent to the microchannel, during at least a part of the initial incubation period.

57. The method of claim 4, wherein the removable barrier comprises an airgap and the removing the removable barrier comprises eliminating the airgap after at least a part of the initial incubation period, comprising using a pipette as a pump to force liquid media exchange between the primary well and the secondary well.

58. The method of claim 1, wherein the detaching comprises exposing the target cell to a dissociation reagent to detach the target cells without detaching the feeder cells, wherein the dissociation reagent comprises EDTA.

59. The method of claim 6, wherein the dissociation reagent comprises a detachment enzyme in a physiological buffer, wherein the detachment enzyme is selected from the group consisting of a trypsin, a collagenase, an elastase, a catalase, a superoxide dismutase, and a dispase.

60. The method of claim 1, wherein the target cells are embedded in a hydrogel dome disposed in the primary well of the well unit in the liquid media, wherein the method further comprises breaking the hydrogel dome prior to the detaching of the target cells.

61. The method of claim 1, wherein the height of the at least one microchannel is in a range of about 10 microns to about 100 microns or about 10 microns to about 75 microns; and wherein the width of the microchannel is in a range of about 150 to about 500 microns, about 200 to about 400 microns, or about 300 microns.
